(19) United States
(12) Patent Application Publication
(10) Pub. No.: US 2025/0157635 A1
(43) Pub. Date: May 15, 2025

(54) PREDICTIONS BASED ON TEMPORAL ASSOCIATED SNAPSHOTS

(71) Applicant: KONINKLIJKE PHILIPS N.V., EINDHOVEN (NL)

(72) Inventors: JOACHIM DIETER SCHMIDT, HAMBURG (DE); XINYU WANG, HAMBURG (DE); FALK UHLEMANN, NORDERSTEDT (DE)

(21) Appl. No.: 18/840,202

(22) PCT Filed: Feb. 22, 2023
(86) PCT No.: PCT/EP2023/054427

§ 371 Date: Aug. 21, 2024

Related U.S. Application Data
(60) Provisional application No. 63/315,227, filed on Mar. 1, 2022.

(51) Int. Cl. G16H 40/20

(52) U.S. Cl. CPC G16H 40/20

(57) ABSTRACT

Methods, apparatuses and systems provide for technology that translates detected physical events to provide information about the current state of a patient process and predict the timing of subsequent states. Events may be decomposed into a series of snapshots associated with timestamps. The embodiments herein determine patterns between the events to identify and predict future states. For example, some embodiments may generate a snapshot stack, and generate a predicted next snapshot based on the snapshot stack.

### FIELD OF THE INVENTION

Embodiments generally relate to predictions based on temporal associated snapshots, and generating predictions about future snapshots (e.g., completion of process steps).

### BACKGROUND OF THE INVENTION

The execution of clinical processes (e.g., radiological imaging), may vary significantly due to unforeseen conditions. This situation is aggravated by the fact that aspects (e.g., duration) of such clinical processes are difficult to predict as no one participant has all the pertinent information or understands how to synthesize such information into a logical estimation of duration. Thus, predictions of such aspects are unreliable resulting in an interrupted and discontinuous workflow. Consequently, scheduling, resource management, and patient management are sub-optimal and degrade the overall performance of an organizational unit.

### SUMMARY OF THE INVENTION

Some embodiments include an event tracking system, comprising a network controller to receive event data from one or more of a sensor or transmitter, and a processor, a memory containing a set of instructions, which when executed by the processor, cause the event tracking system to access a snapshot stack associated with previous events, clone a portion of the snapshot stack, update a first snapshot of the cloned portion based on the event data to generate a modified portion, wherein the first snapshot is associated with the sensor, add the modified portion to the snapshot stack to generate an updated snapshot stack, and predict one or more future snapshots based on the updated snapshot stack.

Some embodiments include at least one computer readable storage medium comprising a set of instructions, which when executed by a computing device, cause the computing device to access a snapshot stack associated with previous events, clone a portion of the snapshot stack, update a first snapshot of the cloned portion based on event data to generate a modified portion, wherein the first snapshot is associated with one or more of a sensor or transmitter, add the modified portion to the snapshot stack to generate an updated snapshot stack, and predict one or more future snapshots based on the updated snapshot stack.

Some embodiments include a method comprising accessing a snapshot stack associated with previous events, cloning a portion of the snapshot stack, updating a first snapshot of the cloned portion based on event data to generate a modified portion, wherein the first snapshot is associated with a sensor or transmitter, adding the modified portion to the snapshot stack to generate an updated snapshot stack, and predicting one or more future snapshots based on the updated snapshot stack.

### BRIEF DESCRIPTION OF THE DRAWINGS

The various advantages of the embodiments of the present disclosure will become apparent to one skilled in the art by reading the following specification and appended claims, and by referencing the following drawings, in which:

FIG. 1 is a diagram of an example of a prediction architecture according to an embodiment;

FIG. 2 is a diagram of an example of a data class events according to an embodiment;

FIG. 3 is a diagram of an example of a data class process definition according to an embodiment;

FIG. 4 is a diagram of an example of a snapshot training process according to an embodiment;

FIGS. 5A, 5B and 5C is a diagram of an example of a collection of training data according to an embodiment;

FIG. 6 is a diagram of an example of a training process according to an embodiment;

FIG. 7 is a diagram of an example of a model according to an embodiment;

FIG. 8 is a diagram of an example of a data flow according to an embodiment;

FIG. 9 is a diagram of an example of a process to generate a series of snapshots according to an embodiment; and

FIG. 10 is a block diagram of an example of an example of a neural network architecture according to an embodiment.

### DETAILED DESCRIPTION OF THE EMBODIMENTS

From a technical point of view, there is a need for a structured and flexible approach that translates detected physical events to provide information about the current state of a patient process and predict the timing of subsequent states. Thus, some embodiments include a “Snapshots-to-Snapshot” approach to solve the aforementioned problems. Events may be decomposed into a series of snapshots associated with timestamps. The embodiments herein determine patterns between the events to identify and predict future states. For example, some embodiments may generate a snapshot stack, and generate a predicted next snapshot based on the snapshot stack.

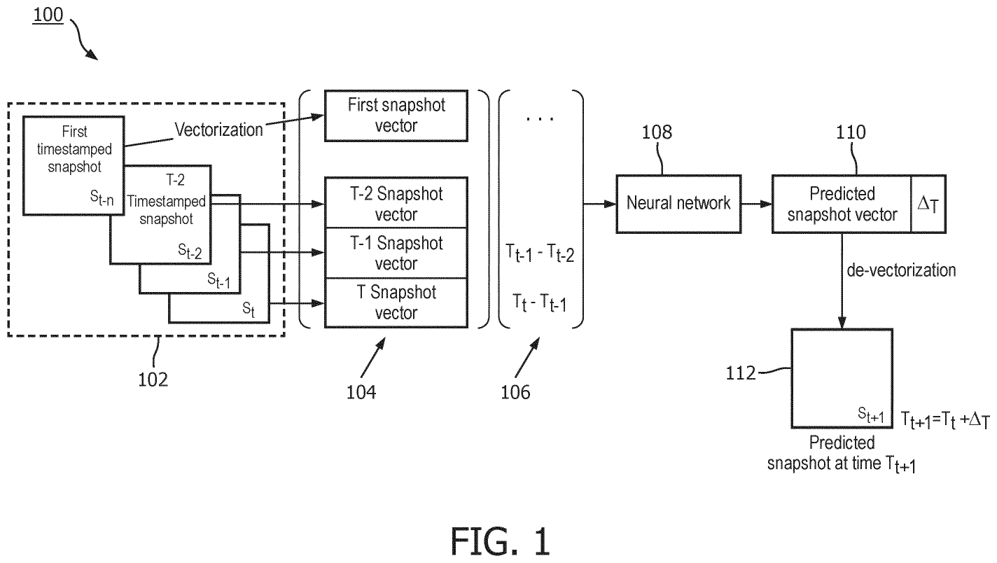

Turning now to FIG. 1, a prediction architecture 100 (e.g., a computing device, server, mobile device, etc.) is illustrated. The prediction architecture 100 may receive events, translate the events into snapshots, store the snapshots into a snapshot stack and generate a prediction and/or action based on the snapshot stack. A series of first-T timestamped snapshots 102 are captured from a plurality of sensors. A domain object model (e.g., an entire data representation of objects in a specific domain) and a vectorized state (e.g., a relatively low-dimensional space which represents high-dimensional vectors) of the domain object model may be referred to as a snapshot. Various models may be used to generate the vectors, such as Word2Vec, Doc2Vec, and fastText. A domain may comprise a specific room, area, or process that is to be modeled. The domain may include all events that are utilized to form a future prediction (explained below).

Vectorization may be utilized in text mining as a preprocessing step so that machine learning algorithms can be applied to various contexts and purposes (e.g., cluster documents into a number of groups, and to further extract topics from each group). In such contexts, vectorization is a process of conversion of a document into a numeric array, using the meaningful words in the collection of documents. Eventually, the collection of documents becomes a matrix, whose single row is one vectorized document. In the present example, vectorization may be the numeric conversion of statuses of concerned sensors and devices.

The first-T timestamped snapshots 102 may originate with (e.g., be captured by) sensors. For example, a first sensor may be a door sensor that senses a state of a door (e.g., whether a door to a CT scanner room is open or closed), and store the state as the first timestamped snapshot Sₜ₋ₙ and other snapshots (e.g., T-2) of the first-T timestamped snapshots 102. A different sensor associated with the first sensor (e.g., may track a same patient process or patient flow) may be a radiation sensor that senses a state of a CT scanner (e.g., whether the CT scanner is scanning), and store the state as the T-2 timestamped snapshot Sₜ₋₂. The other snapshots of the first-T timestamped snapshots 102 may be similarly generated by different sensors or other connected IT systems. Thus, the sensor readings may be indicative of state data that is stored as the first-T timestamped snapshots 102 may be similarly generated by different sensors or IT systems.

In some embodiments and will be explained below, the architecture 100 includes a network controller to receive event data from a plurality of sensors. The architecture 100 (e.g., which includes a controller that comprises hardware logic, configurable logic, and/or a computing device) may access a snapshot stack (e.g., the first-T timestamped snapshots 102) associated with previous events, clone a portion of the snapshot stack, update a first snapshot of the portion of the cloned snapshot stack based on the event data to generate a modified portion, where the first snapshot is associated with the first sensor, add the modified portion to the snapshot stack to generate an updated snapshot stack and predict one or more future snapshots based on the updated snapshot stack.

Each of the first-T timestamped snapshots 102 may comprise readings from multiple sensors. For example, the T timestamped snapshot may include sensors readings from a patient monitor that monitors a position of the patient that is to undergo the CT scan, a door sensor that determines if a door to the CT room is closed or open, etc.

The architecture 100 may vectorize the first-T timestamped snapshots 102 and store first-T snapshot vectors into a matrix 104 and a vector of time deltas 106. The first-T snapshot vectors may be neural network embeddings. An embedding may be a mapping of a discrete variable to a vector of continuous numbers. The matrix 104 and the vector of time deltas 106 may be an underlying event model which represents and stores the state/changes of physical objects (e.g., doors, medical equipment, persons in a room, etc.) and/or inseparable collections thereof (e.g., operation of scanners within proximity, aggregated patient information, etc.) or process information (e.g., accumulated delay, utilization targets, etc.). Thus, the architecture 100 may translate events into state changes of the first-T snapshot vectors. The first-T snapshot vectors forming the matrix 104 and the vector of time deltas 106 may be a snapshot stack that stores time-stamped snapshots of an environment (e.g., a hospital environment).

Each row of the matrix 104 is a vectorized snapshot. Each value in the vector of time deltas 106 is the time difference of two consecutive snapshots. For example, the Tₜ-Tₜ₋₁ time delta may be the difference between a time at which the Sₜ timestamped snapshot (which corresponds to the T snapshot vector) was captured by a sensor (or sensors corresponding to the T snapshot vector), and when a previous timestamped snapshot was captured by the sensor (or sensors corresponding to the T snapshot vector). Each specific time delta of the vector of time deltas 106 is stored in association with the first-T snapshot vectors that corresponds to the specific time delta. For example, the Tₜ-Tₜ₋₁ time delta is stored in association with the T snapshot vector.

A neural network 108 may process the matrix 104 and the vector of time deltas 106. In some embodiments, the neural network 108 may be Long Short-Term Memory (LSTM) Neural Network (NN). A LSTM is a recurrent network architecture that operates in conjunction with an appropriate gradient-based learning algorithm. For example, a LSTM NN may have an adept capability to learn from historical observations, detect the hidden patterns of time-related events and predict future values in a sequence. The neural network 108 may have been previously trained. For example, the neural network 108 may include a machine learning algorithm that is trained with snapshots including object specific and aggregated events, and after training provides predictions on the next snapshot and respective specific object states.

The neural network 108 may detect whether any patterns exist in the matrix 104 and the vector of time deltas 106. The neural network 108 may generate a predicted snapshot vector 110 at a future time Tₜ₊₁. The architecture 100 may de-vectorize the predicted snapshot vector into a predicted snapshot at time Tₜ₊₁. For example, the architecture 100 may translate the predicted snapshot into resources, activities and processes, and integration of translated information with user-friendly interface to inform participants in the workflow.

In some embodiments, the architecture 100 may further take appropriate action based on the predicted snapshot at time Tₜ₊₁ 112. For example, some embodiments may automatically adjust parameters of one or more systems based on the predicted snapshot at time Tₜ₊₁ 112. For example, some embodiments may identify whether an appropriate action (e.g., adjust a temperature, notify other parties of time adjustments, etc.) may be taken based on the predicted snapshot at time Tₜ₊₁ 112. Thus, in some embodiments, the architecture 100 further maps snapshots to process instance states that may be used for communicating process states to users. In some embodiments, the architecture 100 generates one or more of resource related information (e.g., when a CT scanner will be occupied or unoccupied) or activity related interpretation (e.g., whether a room should be cleaned and/or an action to undertake) based on the updated snapshot stack. In some embodiments, the architecture 100 accesses a set of rules that links states of the updated snapshot stack to a resource of interest. In some embodiments, the architecture 100 interpret outputs of the activity updated by changes of one or more snapshots that contain information about resources. In some embodiments, a respective snapshot of the snapshot stack is updated in response to a change to a state of a physical object associated with the respective snapshot (e.g., a sensor may sense that the physical object is changed and adjust the snapshot stack accordingly).

In some embodiments, the architecture 100 generates the snapshot stack based on previous event data from the plurality of sensors, where the previous event data was previously received. The plurality of sensors may be associated with a hospital environment.

As a detailed example, consider Snapshot Stack, such as first-T snapshots 102, that includes n+1 snapshots: Sₜ₋ₙ, Sₜ₋₍ₙ₋₁₎, Sₜ₋₍ₙ₋₂₎, . . . , Sₜ₋₂, Sₜ₋₁, Sₜ, and these snapshots are attached with timestamps Tₜ₋ₙ, Tₜ₋₍ₙ₋₁₎, Tₜ₋₍ₙ₋₂₎, . . . , Tₜ₋₂, Tₜ₋₁, Tₜ with Sₜ₋ₙ denoting the first snapshot with timestamp Tₜ₋ₙ, and Sₜ denoting the last snapshot with timestamp Tₜ. The subscripts t-n, t-(n-1), t-(n-2), . . . , t-2, t-1 denote a retrospective (e.g., occurred in the past) meaning. The subscript (e.g., “t”) denotes the current point of time. Sₜ₊₁ (e.g., the exact event of Sₜ₊₁) and the time difference between Sₜ and Sₜ₊₁ may be intended to be predicted by the neural network 108.

The number of objects in the observed domain (e.g., the number of sensors or scanners) is fixed through the snapshot stack, but the statuses of the number of objects are updated through the first to the last snapshots upon upcoming events that trigger the changes of objects. For example, if there is only a door sensor and a CT scanner in the domain, the snapshot stack may be updated just before any CT exam takes place at the beginning of day (e.g., 8:00 AM). Then in the first snapshot Sₜ₋ₙ some embodiments may initialize the status of the door as being set to ‘closed’ and the CT scanner is ‘idle’, denoted by Sₜ₋ₙ={door-closed, CT=idle} and with timestamp Tₜ₋ₙ=8:00 AM.

After five minutes, a patient and a nurse enter the scanner room, then some embodiments update the status of door to be ‘open’ and the CT scanner is ‘idle’ in the 2^{nd} snapshot Sₜ₋₍ₙ₋₁₎={door=open, CT=idle} with Tₜ₋₍ₙ₋₁₎=8:05 AM. After three minutes, the patient is placed on the table, the CT scanner is ready to be operated, and the nurse comes out of the scanner room leaving the door closed, the 3^{rd} snapshot becomes Sₜ₋₍ₙ₋₂₎={door=closed, CT=‘ready’} with Tₜ₋₍ₙ₋₂₎=8:08.

Two minutes later, the CT scanner starts scanning the patient, the status of the CT scanner is updated to ‘running’ and the door is ‘closed’, so in the 4^{th} snapshot Sₜ₋₍ₙ₋₃₎={door=closed, CT=running} with timestamp Tₜ₋₍ₙ₋₃₎=8:10 AM. Suppose after fifteen minutes, while the CT scanner completes all the scans, the 5th snapshot becomes Sₜ={door=closed, CT=scans completed} with Tₜ=8:25. At this point of time, it may be advantageous to determine at what time the patient will leave the scanner room. Thus, the neural network 108 may predict the time at which the patient will leave the room.

Thus, in this example, we have a Snapshot Stack of 5 snapshots, thus n=4 ranging from n=0 to n=4. Specifically, the snapsnot stack is illustrated by the table below. Before feeding the neural network 108, embodiments vectorize each snapshot into a vector denoted by the matrix 104 and the vector of time deltas 106. For each of these vectors (except the 1st vector for n=0), embodiments may append the time difference between the different snapshots. The result of the neural network 108 is the 6th snapshot, Sₜ₊₁={door-open, CT=idle} and the time difference to the 5th snapshot (e.g., 4 minutes). In this way, embodiments may predict in advance that the patient would leave the scanner room at 8:29 AM, and the scanner room will be vacant and ready for next exam.

TABLE I

| Snapshot | | | |
|---|---|---|---|
| index | Snapshot notation | Timestamp | Time difference |
| 1st | Sₜ₋₄ = {door = closed, | Tₜ₋₄ = 8:00 | — |
| | CT = idle} | | |
| 2nd | Sₜ₋₃ = {door = open, | Tₜ₋₃ = 8:05 | 5 |
| | CT = idle} | | |
| 3rd | Sₜ₋₂ = {door = closed, | Tₜ₋₂ = 8:08 | 3 |
| | CT = ready to run} | | |
| 4th | Sₜ₋₁ = {door = closed, | Tₜ₋₁ = 8:10 | 2 |
| | CT = running} | | |
| 5th | Sₜ = {door = closed, | Tₜ = 8:25 | 15 |
| | CT = scans completed} | | |

A vectorization example based on the above example is now described. In this example, snapshots are constructed based on the states of the scanner room door and the CT scanner. The door has statuses “closed” and “open,” and the CT scanner has statuses of “idle”, “ready to run”, “running” and “completed.” Embodiments may compose a binary vector from values of 0 and 1 to encode a snapshot at one point of time. 1 denotes the activeness or presence of one status. The vector of the matrix 104 for Table I is illustrated as below in Table II with the corresponding snapshot indices (e.g., the vector of snapshot index 1 of Table II corresponds to the entry of snapshot index 1 of Table I).

TABLE II

| Snapshot | Door | | CT | | | |
|---|---|---|---|---|---|---|
| index | Closed | Open | Idle | ReadyToRun | Running | Completed |
| 1st | 1 | 0 | 1 | 0 | 0 | 0 |
| 2nd | 0 | 1 | 1 | 0 | 0 | 0 |
| 3rd | 1 | 0 | 0 | 1 | 0 | 0 |
| 4th | 1 | 0 | 0 | 0 | 1 | 0 |
| 5th | 1 | 0 | 0 | 0 | 0 | 1 |

Moreover, triggering an automatic action based on the predicted result is possible, and may be based on software and hardware infrastructure. For example, continuing with the example above, suppose that the neutral network predicts that, at 8:29 AM, the patient leaves the scanner room, and the scanner room is vacant, two actions could be triggered automatically. Firstly, if the patient needs mobility assistance to use (e.g., a movable bed or wheelchair), an automatic notification could be sent out to notify the responsible nurse of the predicted meeting time with the patient. So, the nurse may be present at the predicted time with a movable bed or wheelchair to avoid patient waiting. Secondly, the message of ‘scanner room will be vacant at 8:29 am’ may be sent automatically to cleaning personal, who comes to disinfect and clean the scanner and the room without delay. In another example, an automatic cleaning process with robots may be actuated based on the indication that the scanner room is vacant, or automatic power saving features may be enacted such as turning off all lights and unnecessary resources in the scanner room. These actions include a communication system and device integrated into the infrastructure, which hosts applications of embodiments as described herein.

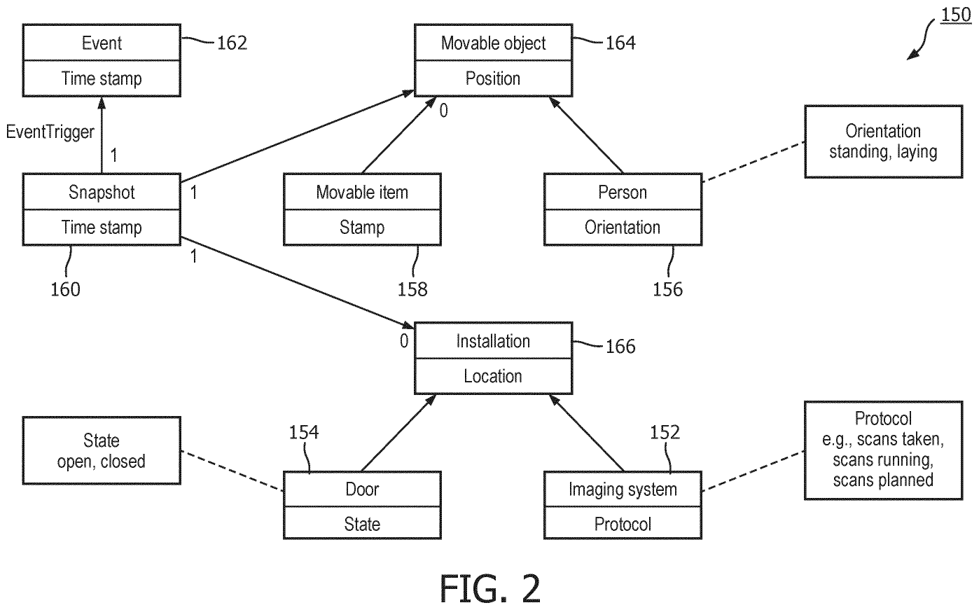

Turning now to FIG. 2, data class events 150 of a snapshot are illustrated. The data class events 150 may correspond to an event (e.g., a single event) as described with reference to architecture 100 (FIG. 1). The data class events 150 may be readily implemented in conjunction with the prediction architecture 100 (FIG. 1). An event may be an occurrence that happens during a process. The data class events 150 reflect the flow of the process and may have a cause or an impact on the process. In some embodiments, events of the data class events 150 are timestamped actions extracted from log files of medical and/or information technology equipment, door state switches, cameras that are used to identify objects in a location, sensors that pick up RF radiation from MRI scanners, and microphones that pick up and analyze sounds in a location. Table III provides examples of events:

TABLE III

| Event Source | Event |
|---|---|
| Door Switch | Door is closed |
| Door Switch | Door is opened |
| MRI system log file | Protocol is selected |
| MRI system log file | Several scans are added to protocol |
| MRI system log file | One scan started |
| MRI system log file | One scan completed |
| Radio Frequency Sensor (RF- | MRI system started scanning |
| Sensor) | |
| Radio Frequency Sensor (RF- | MRI system stopped scanning |
| Sensor) | |
| Camera | Persons are in the MRI Bay |
| Camera | A person is lying on the MRI table |
| Camera | No person is lying on the MRI table |

The data class events 150 may be a snapshot model that is a Domain Object Model, in which objects represent real-world items or a collection thereof. Snapshot 160 includes a plurality of objects described below. Some exemplary objects are medical equipment, such as an imaging system 152, door 154, person 156, movable item 158 (e.g., radio-frequency coils of a magnetic-resonance imaging scanner). Each of these objects has a state that is pertinent to the state of a process or one or more of its activities and are stored as part of the snapshot 160. The object state is updated upon receiving an event from an attached source.

In this example, the snapshot 160 has a timestamp and an event that caused the creation of the snapshot 160. Thus, the snapshot 160 is generated in response to an event 162 being sensed. The snapshot 160 may include a movable object 164 that has a certain position that is tracked when the snapshot 160 is created. The movable object 164 may include a person 156 (at a specific orientation) and a movable item 158 (e.g., a movable surface coil such as for an ankle, knee or head for MR imaging, magnetic resonance imaging item, etc. that is in a specific state). The snapshot 160 includes an installation 166 that includes the door 154 at a state (e.g., open or closed), and the imaging system 152 that is at a protocol. Thus, the snapshot 160 may include diverse sensor readings that are all related to the event 162.

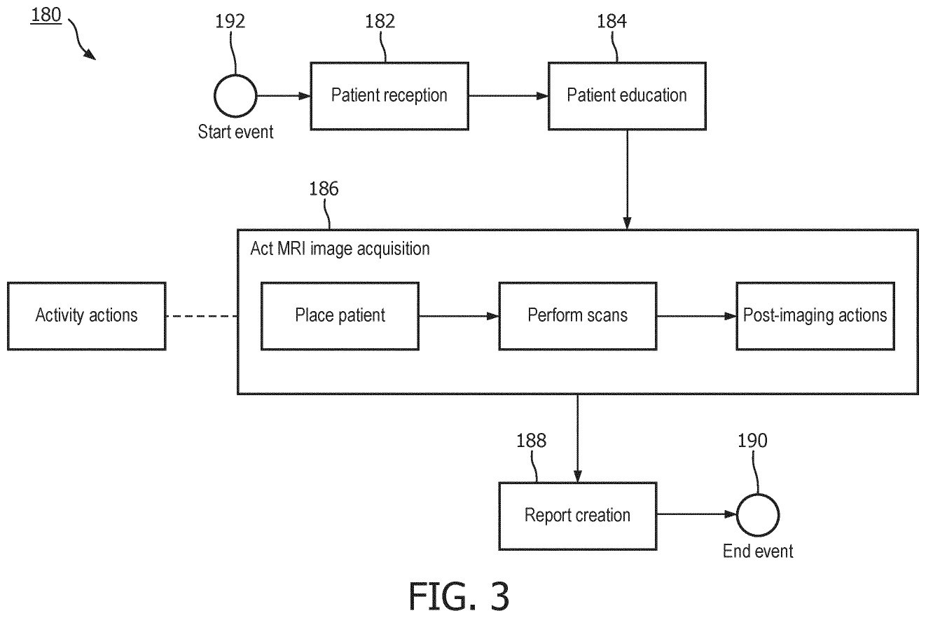

FIG. 3 represents a data class process definition 180 which is a formal representation of a process that may be tracked to predict outcomes. The data class process definition 180 may be readily implemented in conjunction with the prediction architecture 100 (FIG. 1) and/or data class events 150 (FIG. 2). Initially, a start event 192 results in patient reception 182 and patient education 184. An MRI image acquisition 186 includes patient placement, performance of scans and post-imaging actions which leads to a report creation 188 and finally an end event 190.

In some embodiments, a neural network architecture may generate a process instance state (e.g., a snapshot) based on the data class process definition 180. For example, the process instance state may contain information such as resource state (e.g., MRI scanner in MRI Bay 1 is currently performing scan 3 of 6 of protocol “Brain”). The activity with actions states for example that the activity of MRI Image Acquisition is in the state where the patient has been positioned for scanning and scan 3 of 6 of protocol “Brain” is being performed. As an example, activity states may include that the MRI image process has completed patient registration, patient education, and is now conducting the activity MRI Image Acquisition which is in the state of performing scan 3 of 6 of protocol “Brain.”

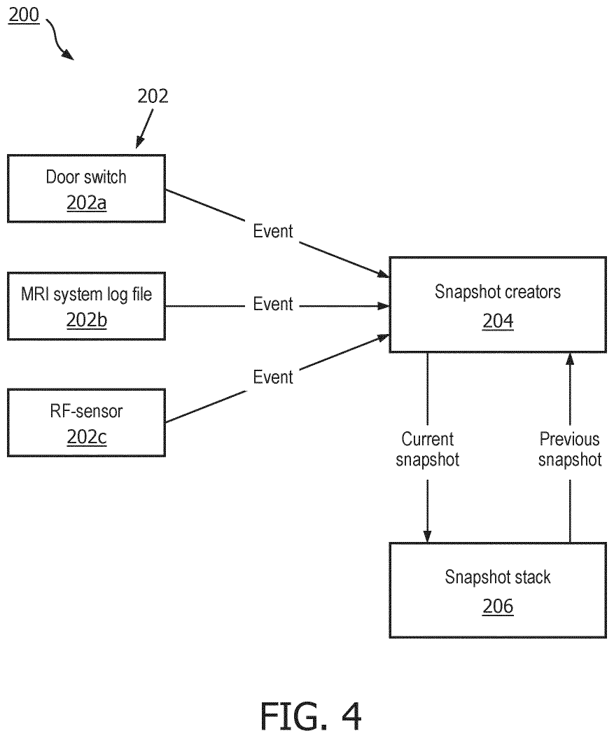

FIG. 4 illustrates a data collection process 200 to collect data for training of a prediction model according to embodiments herein. For example, the prediction model may be trained to predict future states. The snapshot training process 200 may be readily implemented in conjunction with the prediction architecture 100 (FIG. 1), data class events 150 (FIG. 2) and/or data class process definition 180 (FIG. 3). That is, FIG. 4 illustrates a data flow during collection of training data for the prediction model. Events are received from various sources 202 including door switch 202a, MRI system log file 202b and RF-sensor 202c. The snapshot creators 204 translates the events into snapshots, and the snapshots are stored in a snapshot stack 206. The snapshot stack 206 is initialized with an initial snapshot, that contains the domain objects that are used by a system. The initial snapshot is customized to the site where the system (e.g., prediction model) is being used. The initial snapshot may define all installations and movable objects that are considered relevant for operation, and for the training of the machine learning algorithm. The state of all initial objects is set to “Unknown”. The training data may be used for training a neural network to operate as described herein.

In some embodiments, the snapshot training process 200 generates time stamps from time measurements from training event data (e.g., event data from various sources 202) associated with training events. In such embodiments, the snapshot training process 200 vectorizes the training event data into a plurality of vectors, stores the plurality of vectors in association with the timestamps into a matrix, detects patterns between the training events based on the plurality of vectors and the timestamps and predict the one or more future snapshots based on the patterns.

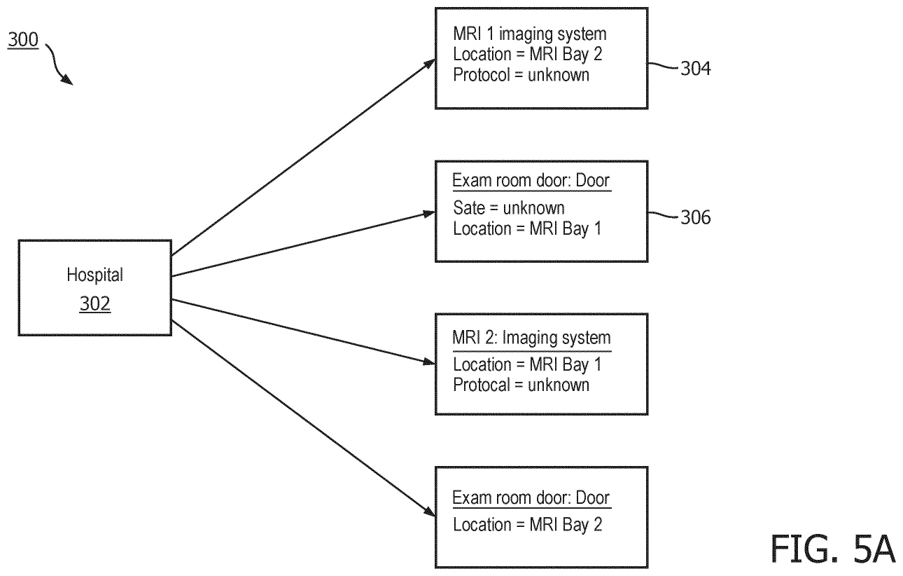

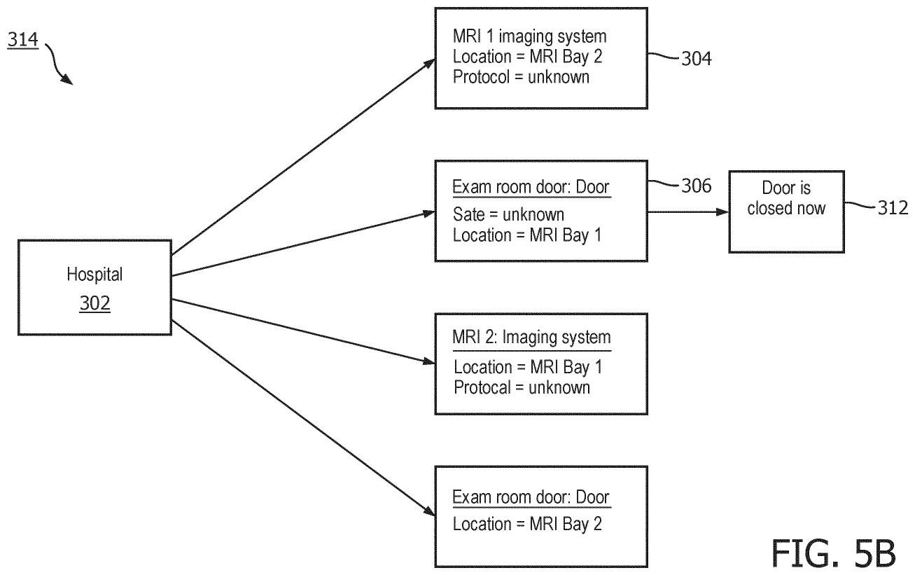

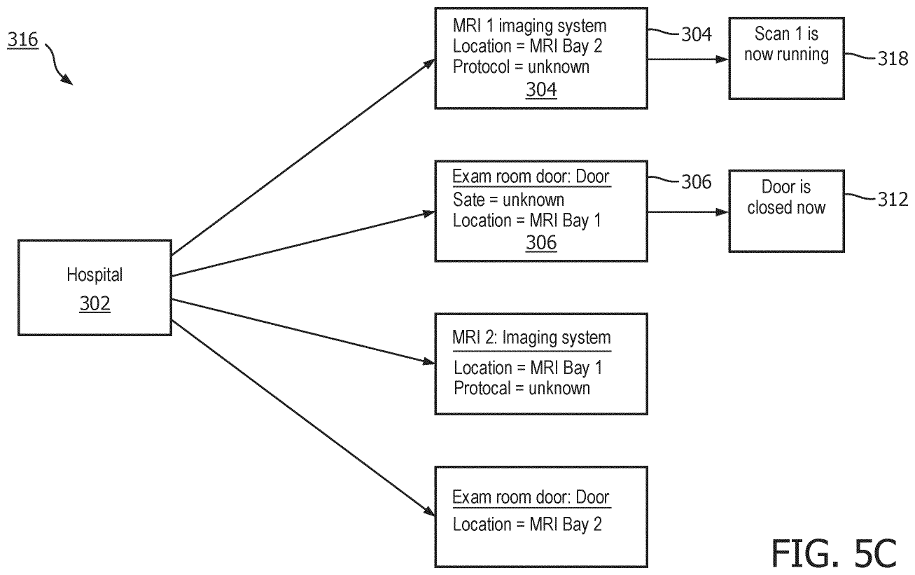

FIGS. 5A, 5B and 5C illustrate a series of snapshots 300, 314, 316 at different times in hospital 302. The series of snapshots 300, 314, 316 may be readily implemented in conjunction with the prediction architecture 100 (FIG. 1), data class events 150 (FIG. 2), data class process definition 180 (FIG. 3) and/or collection of snapshot training process 200 (FIG. 4). An initial snapshot 300 is illustrated in FIG. 5A. When an event is received, an architecture may include a snapshot creator that clones the last snapshot of the snapshot stack, updates the objects (states of the objects), and adds the updated objects as current snapshot to the snapshot stack. As one example, the event “door closed” from exam room door 306 (e.g., via a switch sensor) in MRI Bay 1 indicates a closed door. The resulting second snapshot 314 is shown in FIG. 5B. As shown in the second snapshot 314, the exam room door 306 has now been updated to indicate that the door is now closed 312. Notably, the initial snapshot 300 may be cloned and modified to generate the second snapshot 314. The second snapshot 314 may be added to a snapshot stack that includes the initial snapshot 302.

FIG. 5C illustrates a third snapshot 316. That is, the MRI 1 imaging system 304 may actuated. The second snapshot 314 (the most recent snapshot) may be cloned and modified to generate the third snapshot 316. That is, the second snapshot 314 may be cloned and modified to indicate that an RF-Sensor in MRI Bay 1 is running a scan to generate the third snapshot 316. Thus, the MRI 1 imaging system 304 may be updated to indicate that scan 1 is now running 318.

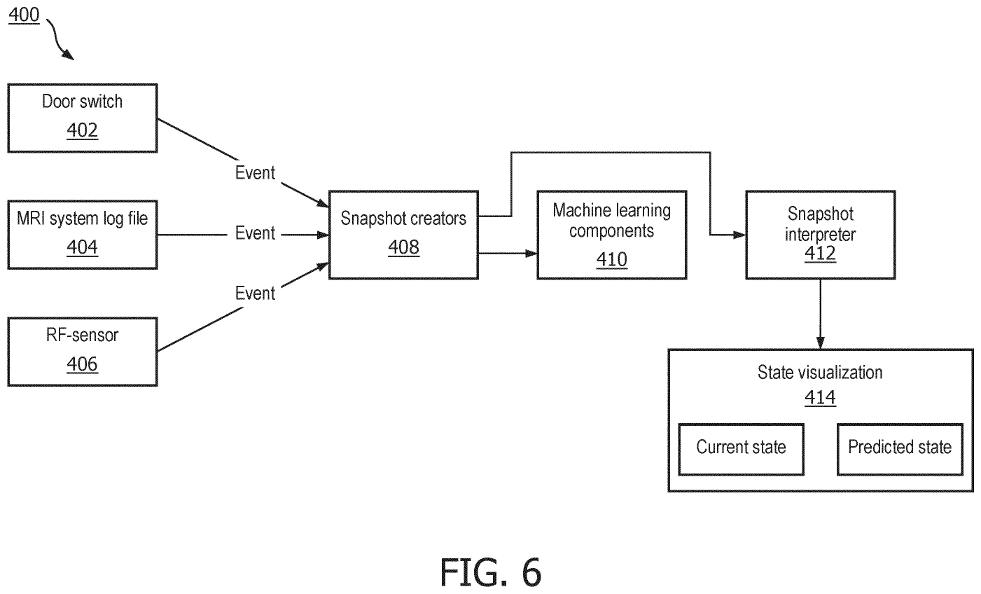

FIG. 6 illustrates a training process 400 for a neural network. The training process 400 may be readily implemented in conjunction with the prediction architecture 100 (FIG. 1), data class events 150 (FIG. 2), data class process definition 180 (FIG. 3), snapshot training process 200 (FIG. 4) and/or series of snapshots 300, 314, 316 (FIGS. 5A, 5B, 5C). The training process 400 includes receiving events from a door switch 402, MRI system log file 404 and RF-sensor 406. Snapshot creators 408 may generate current snapshots and feeds the current snapshots to both a machine learning component 410 (e.g., part of a neural network) and a snapshot interpreter 412 (e.g., another part of the neural network). A state visualization 414 may determine a current state and a predicted state and provide the current state and the predicted state to the state visualization 414. The snapshot interpreter 412 may output a state of resources (e.g., an MRI bay in use and how long it will be in use). For this purpose, the snapshot interpreter 412 includes a set of rules that link the state of snapshot objects to a resource of interest. The snapshot interpreter 412 outputs states of the activity updated by changes of snapshots that contain information about resources (e.g., a camera and/or an MRI scanner). The snapshot interpreter 412 has a set of rules that link the state of snapshot objects to an activity of interest.

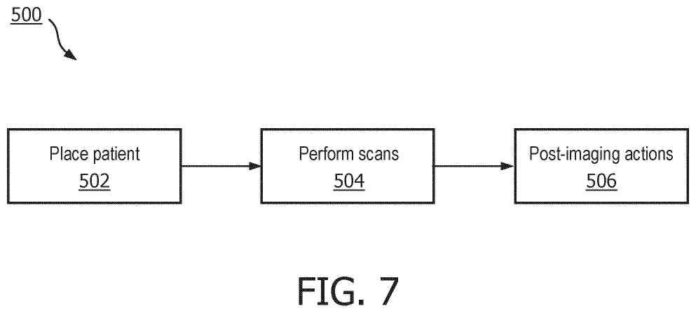

FIG. 7 illustrates a model 500 that may be used for activity related training. The model 500 may be readily implemented in conjunction with the prediction architecture 100 (FIG. 1), data class events 150 (FIG. 2), data class process definition 180 (FIG. 3), snapshot training process 200 (FIG. 4), series of snapshots 300, 314, 316 (FIGS. 5A, 5B, 5C) and/or training process 400 (FIG. 6). In detail, the model 500 may include placement of a patient 502, performance of scans 504 and post-imaging actions 506. For example, the training process 400 (FIG. 6) may train the machine learning components 410 and the snapshot interpreter 412 based on the model 500.

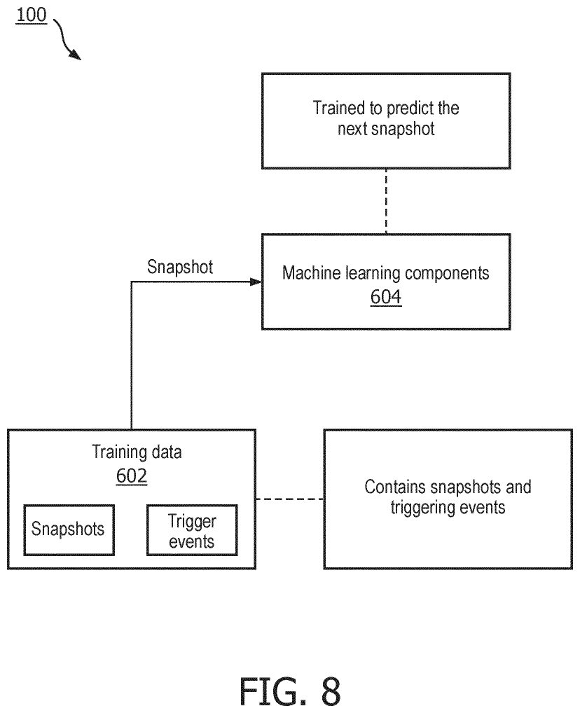

FIG. 8 illustrates data flow 600 during a machine learning model training. The data flow 600 may be readily implemented in conjunction with the prediction architecture 100 (FIG. 1), data class events 150 (FIG. 2), data class process definition 180 (FIG. 3), snapshot training process 200 (FIG. 4), series of snapshots 300, 314, 316 (FIGS. 5A, 5B, 5C), training process 400 (FIG. 6) and/or model 500 (FIG. 7). The machine learning model training includes training data 602 which includes snapshots and trigger events. The machine learning components 604 may be trained based on the training data 602. The machine learning components 604 may be trained to predict a next snapshot.

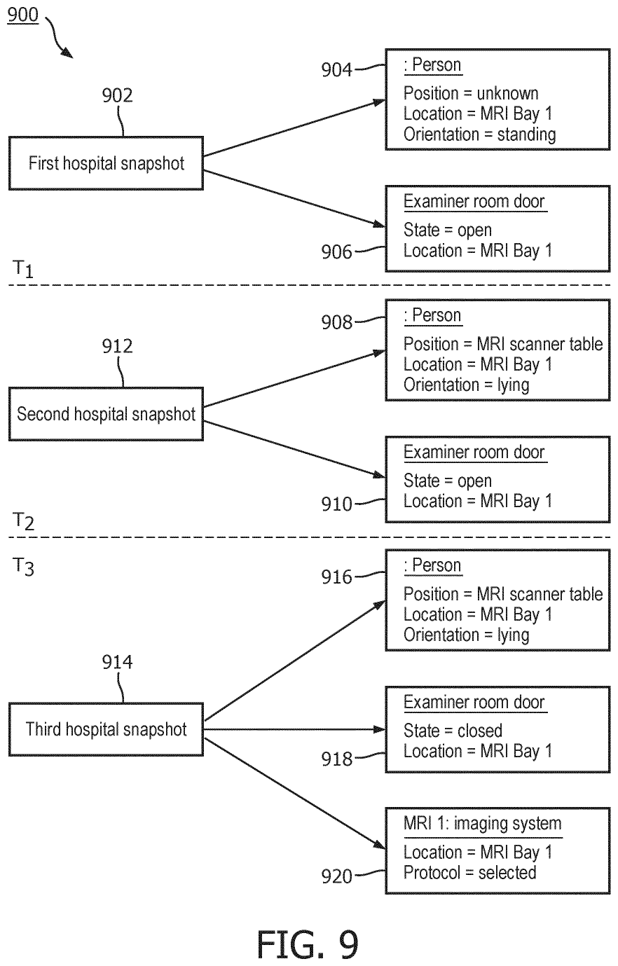

FIG. 9 illustrates a process 900 to generate a series of snapshots. The process 900 may be readily implemented in conjunction with the prediction architecture 100 (FIG. 1), data class events 150 (FIG. 2), data class process definition 180 (FIG. 3), snapshot training process 200 (FIG. 4), series of snapshots 300, 314, 316 (FIGS. 5A, 5B, 5C), training process 400 (FIG. 6), model 500 (FIG. 7) and/or data flow 600 (FIG. 8). At time T₁, the process 900 determines a snapshot interpretation based on first hospital snapshot 902 to detect the start of activity “Place Patient” based on activities 904, 906. That is, activity 904 includes a person who is oriented in a standing position, and activity 906 includes an examiner room door being open. At time T₂, the second hospital snapshot 912 reflects that the process 900 determines that the snapshot interpretation detects the end of the activity “Place Patient” based on activities 908, 910. That is, activity 908 indicates that the person is now lying down and activity 910 indicates that the examiner room door is open. At time T₃, the process 900 determines that a third hospital snapshot 914 includes a snapshot interpretation to detect the start of activity “Perform Scans” for MRI bay 1 based on events 916, 918, 920.

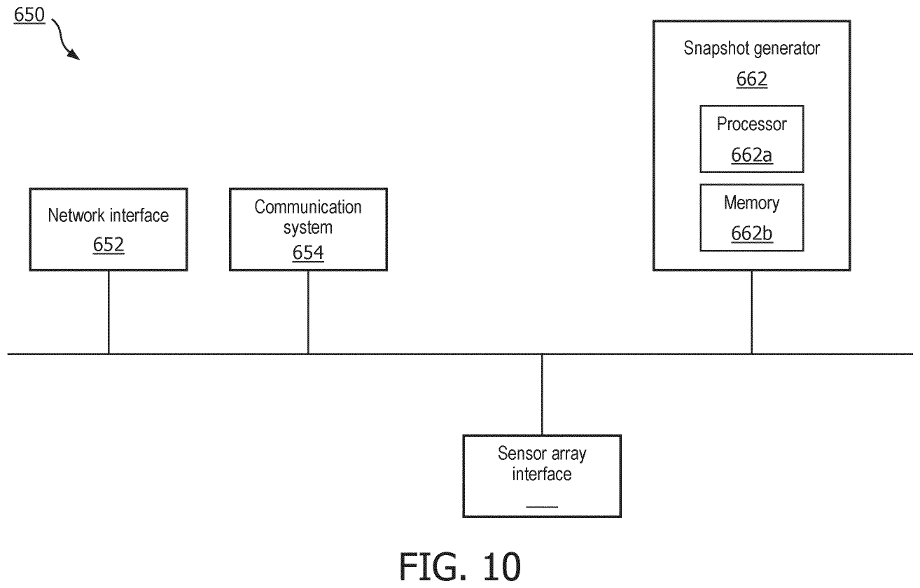

FIG. 10 shows a more detailed example of a neural network architecture 650. The neural network architecture 650 may be readily implemented in conjunction with the prediction architecture 100 (FIG. 1), data class events 150 (FIG. 2), data class process definition 180 (FIG. 3), snapshot training process 200 (FIG. 4), series of snapshots 300, 314, 316 (FIGS. 5A, 5B, 5C), training process 400 (FIG. 6), model 500 (FIG. 7), data flow 600 (FIG. 8) process 900 (FIG. 9).

In the illustrated example, the neural network architecture 650 may include a network interface system 652, a communication system 654, and a sensor array interface 668. The sensor array interface 668 may interface with a plurality of sensors, for example door sensors, switch sensors, imaging sensors, or other connected (IT) systems etc. The sensor array interface 668 may interface with any type of sensor or event data transmitting system suitable for operations as described herein.

A snapshot generator 662 may receive data from the sensor array interface 668. The snapshot generator 662 may analyze events, generate snapshots and predict a next snapshot. The predict snapshot may be provided to a communication system 654 that communicates with one or more other computing devices.

The snapshot generator 662 may include a processor 662a (e.g., embedded controller, central processing unit/CPU) and a memory 662b (e.g., non-volatile memory/NVM and/or volatile memory). The memory 662b contains a set of instructions, which when executed by the processor 662a, cause the snapshot generator 662 to operate as described herein.

Further, the disclosure comprises additional examples as detailed in the following Examples below.

Example 1. An event tracking system, comprising:
a network controller to receive event data from one or more of a sensor or transmitter;
a processor; and
a memory containing a set of instructions, which when executed by the processor, cause the event tracking system to:
   access a snapshot stack associated with previous events;
   clone a portion of the snapshot stack;
   update a first snapshot of the cloned portion based on the event data to generate a modified portion, wherein the first snapshot is associated with the sensor;
   add the modified portion to the snapshot stack to generate an updated snapshot stack; and
predict one or more future snapshots based on the updated snapshot stack.

Example 2. The event tracking system of Example 1, wherein the set of instructions, which when executed by the processor, cause the event tracking system to:
generate one or more of resource related information or activity related interpretation based on the updated snapshot stack.

Example 3. The event tracking of Example 1, wherein the set of instructions, which when executed by the processor, cause the event tracking system to:
vectorize the event data to generate a vector;
identify a time stamp associated with the event data; and
store the first snapshot to include the vector and the time stamp as part of the modified portion.

Example 4. The event tracking system of Example 1, wherein the set of instructions, which when executed by the processor, cause the event tracking system to:
update the first snapshot of the portion in response to a change to a state of a physical object associated with the first snapshot.

Example 5. The event tracking system of Example 1, wherein the set of instructions, which when executed by the processor, cause the event tracking system to:
predict the one or more future snapshots with a Long Short-Term Memory neural network.

Example 6. The event tracking system of Example 1, wherein the sensor or transmitter is associated with a hospital environment.

Example 7. The event tracking system of any one of Examples 1 to 6, wherein the set of instructions, which when executed by the processor, cause the event tracking system to:
generate time stamps from time measurements from training event data associated with training events;
vectorize the training event data into a plurality of vectors;
store the plurality of vectors in association with timestamps into a matrix;
detect patterns between the training events based on the plurality of vectors and the timestamps; and
predict the one or more future snapshots based on the patterns.

Example 8. At least one computer readable storage medium comprising a set of instructions, which when executed by a computing device, cause the computing device to:
access a snapshot stack associated with previous events;
clone a portion of the snapshot stack;
update a first snapshot of the cloned portion based on event data to generate a modified portion, wherein the first snapshot is associated with one or more of a sensor or transmitter;
add the modified portion to the snapshot stack to generate an updated snapshot stack; and
predict one or more future snapshots based on the updated snapshot stack.

Example 9. The at least one computer readable storage medium of Example 8, wherein the instructions, when executed, cause the computing device to:
generate one or more of resource related information or activity related interpretation based on the updated snapshot stack.

Example 10. The at least one computer readable storage medium of Example 8, wherein the instructions, when executed, cause the computing device to:
vectorize the event data to generate a vector;
identify a time stamp associated with the event data; and
store the first snapshot to include the vector and the time stamp as part of the modified portion.

Example 11. The at least one computer readable storage medium of Example 8, wherein the instructions, when executed, cause the computing device to:
update the first snapshot in response to a change to a state of a physical object associated with the first snapshot.

Example 12. The at least one computer readable storage medium of Example 8, wherein the instructions, when executed, cause the computing device to:
predict the one or more future snapshots with a Long Short-Term Memory neural network.

Example 13. The at least one computer readable storage medium of Example 8, wherein the sensor is associated with a hospital environment.

Example 14. The at least one computer readable storage medium of any one of Examples 8 to 13, wherein the instructions, when executed, cause the computing device to:
generate time stamps from time measurements from training event data associated with training events;
vectorize the training event data into a plurality of vectors;
store the plurality of vectors in association with the time stamps into a matrix;
detect patterns between the training events based on the plurality of vectors and the time stamps; and
predict the one or more future snapshots based on the patterns.

Example 15. A method comprising:
accessing a snapshot stack associated with previous events;
cloning a portion of the snapshot stack;
updating a first snapshot of the cloned portion based on event data to generate a modified portion, wherein the first snapshot is associated with a sensor or transmitter;
adding the modified portion to the snapshot stack to generate an updated snapshot stack; and
predicting one or more future snapshots based on the updated snapshot stack.

Example 16. The Example of claim 15, further comprising:
generating one or more of resource related information or activity related interpretation based on the updated snapshot stack.

Example 17. The Example of claim 15, further comprising:
vectorizing the event data to generate a vector;
identifying a time stamp associated with the event data; and
storing the first snapshot to include the vector and the time stamp as part of the modified portion.

Example 18. The Example of claim 15, further comprising:
updating the first snapshot of the portion in response to a change to a state of a physical object associated with the first snapshot.

Example 19. The Example of claim 15, further comprising:
predicting the one or more future snapshots with a long short-term memory neural network.

Example 20. The Example of any one of claims 15 to 19, further comprising:
generating time stamps from time measurements from training event data associated with training events;
vectorizing the training event data into a plurality of vectors;
storing the plurality of vectors in association with the time stamps into a matrix;
detecting patterns between the training events based on the plurality of vectors and the time stamps; and
predicting the one or more future snapshots based on the patterns.

The above described methods and systems may be readily combined together if desired. The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

Those skilled in the art will appreciate from the foregoing description that the broad techniques of the embodiments of the present disclosure can be implemented in a variety of forms. Therefore, while the embodiments of this disclosure have been described in connection with particular examples thereof, the true scope of the embodiments of the disclosure should not be so limited since other modifications will become apparent to the skilled practitioner upon a study of the drawings, specification, and following claims.

We claim:

1. An event tracking system, comprising:
a network controller to receive event data from one or more of a sensor or transmitter;
a processor; and
a memory containing a set of instructions, which when executed by the processor, cause the event tracking system to:
access a snapshot stack associated with previous events;
clone a portion of the snapshot stack;
update a first snapshot of the cloned portion based on the event data to generate a modified portion, wherein the first snapshot is associated with the one or of the sensor or transmitter;
add the modified portion to the snapshot stack to generate an updated snapshot stack; and
predict one or more future snapshots based on the updated snapshot stack.

2. The event tracking system of claim 1, wherein the set of instructions, which when executed by the processor, cause the event tracking system to:
generate one or more of resource related information or activity related interpretation based on the updated snapshot stack.

3. The event tracking of claim 1, wherein the set of instructions, which when executed by the processor, cause the event tracking system to:
vectorize the event data to generate a vector;
identify a time stamp associated with the event data; and
store the first snapshot to include the vector and the time stamp as part of the modified portion.

4. The event tracking system of claim 1, wherein the set of instructions, which when executed by the processor, cause the event tracking system to:
update the first snapshot of the portion in response to a change to a state of a physical object associated with the first snapshot.

5. The event tracking system of claim 1, wherein the set of instructions, which when executed by the processor, cause the event tracking system to:
predict the one or more future snapshots with a Long Short-Term Memory neural network.

6. The event tracking system of claim 1, wherein the sensor or transmitter is associated with a hospital environment.

7. The event tracking system of claim 1, wherein the set of instructions, which when executed by the processor, cause the event tracking system to:
generate time stamps from time measurements from training event data associated with training events;
vectorize the training event data into a plurality of vectors;
store the plurality of vectors in association with timestamps into a matrix;
detect patterns between the training events based on the plurality of vectors and the timestamps; and
predict the one or more future snapshots based on the patterns.

8. At least one computer readable storage medium comprising a set of instructions, which when executed by a computing device, cause the computing device to:
access a snapshot stack associated with previous events;
clone a portion of the snapshot stack;
update a first snapshot of the cloned portion based on event data to generate a modified portion, wherein the first snapshot is associated with one or more of a sensor or transmitter;
add the modified portion to the snapshot stack to generate an updated snapshot stack; and
predict one or more future snapshots based on the updated snapshot stack.

9. The at least one computer readable storage medium of claim 8, wherein the instructions, when executed, cause the computing device to:
generate one or more of resource related information or activity related interpretation based on the updated snapshot stack.

10. The at least one computer readable storage medium of claim 8, wherein the instructions, when executed, cause the computing device to:
vectorize the event data to generate a vector;
identify a time stamp associated with the event data; and
store the first snapshot to include the vector and the time stamp as part of the modified portion.

11. The at least one computer readable storage medium of claim 8, wherein the instructions, when executed, cause the computing device to:
update the first snapshot in response to a change to a state of a physical object associated with the first snapshot.

12. The at least one computer readable storage medium of claim 8, wherein the instructions, when executed, cause the computing device to:
predict the one or more future snapshots with a Long Short-Term Memory neural network.

13. The at least one computer readable storage medium of claim 8, wherein the sensor or the transmitter is associated with a hospital environment.

14. The at least one computer readable storage medium of claim 8, wherein the instructions, when executed, cause the computing device to:
generate time stamps from time measurements from training event data associated with training events;
vectorize the training event data into a plurality of vectors;
store the plurality of vectors in association with the time stamps into a matrix;
detect patterns between the training events based on the plurality of vectors and the time stamps; and
predict the one or more future snapshots based on the patterns.

15. A method comprising:
accessing a snapshot stack associated with previous events;
cloning a portion of the snapshot stack;
updating a first snapshot of the cloned portion based on event data to generate a modified portion, wherein the first snapshot is associated with a sensor or transmitter;
adding the modified portion to the snapshot stack to generate an updated snapshot stack; and
predicting one or more future snapshots based on the updated snapshot stack.
